(19) United States
(12) Patent Application Publication
(10) Pub. No.: US 2009/0320850 A1
(43) Pub. Date: Dec. 31, 2009

(54) RESPIRATION SYSTEM

(75) Inventors: Oliver WALLNEWITZ, Lübeck (DE); Krisztina SOLTÈSZ, Lübeck (DE)

Correspondence Address: MCGLEW & TUTTLE, PC, P.O. BOX 9227, SCARBOROUGH STATION, SCARBOROUGH, NY, 10510-9227, US

(73) Assignee: Dräger Madical AG & Co. KG, Lübeck (DE)

(21) Appl. No.: 12/489,698

(22) Filed: Jun. 23, 2009

(30) Foreign Application Priority Data
Jun. 28, 2008 (DE) 10 2008 030 789.0

(51) Int. Cl. A61M 16/06

(52) U.S. Cl. 128/207.11

(57) ABSTRACT

A respiration system (1) is provided with a breathing mask (2), a connection device (3) embodied on the breathing mask (2) for connecting the breathing mask (2) to a flexible air tube (15), a forehead support (4), and a band system (5) for attaching the breathing mask (2) to a head of a patient. An attaching device (9a, 9b, 9c, 9d) is arranged on the breathing mask (2) and on the forehead support (4) for the band system (5). The respiration system (1) is able to be adapted to the greatest variety of anatomies and requirements of a patient's head. Further, the respiration system (1) is simple and reliable to use and has low manufacturing costs. The forehead support (4) can be detachably connected to the breathing mask (2), so that the respiration system (1) can be used for respirating patients both with and without a forehead support (4).

### CROSS REFERENCE TO RELATED APPLICATIONS

This application claims the benefit of priority under 35 U.S.C. § 119 of German Patent Application 10 2008 030 789.0 filed Jun. 28, 2008, the entire contents of which are incorporated herein by reference.

### FIELD OF THE INVENTION

The present invention pertains to a respiration system (also known as ventilation system) with a breathing mask, a connection device embodied on the breathing mask for connecting the breathing mask to a flexible air tube, a forehead support, a band system for attaching the breathing mask to a head of a patient and attaching elements for the band system arranged on the breathing mask and on the forehead support.

### BACKGROUND OF THE INVENTION

Respiration systems are used for noninvasive respiration of patients. Respiration systems comprise a breathing mask that is placed on the face area of the patient and generally covers both the nose and mouth, so that the patient can be supplied with artificial breathing air when breathing through the nose and mouth.

DE 103 38 169 A1 shows a respiration system that is representative of a class of respiration systems, which comprises a forehead support arranged on the breathing mask. The forehead support can be attached in various positions through a slot provided with catches. As a drawback, with the respiration system, an adaptation to different anatomies of the head of the patient is possible only with limitations. Furthermore, it is not possible to dispense with the forehead support, for example, in patients with a head injury.

A respiration system of the present class is known from US 2003/0034034 A1. A forehead support is pivotably connected to a breathing mask, such that the above-described drawbacks occur here as well.

Respiration systems with and without forehead support are known. To respirate a patient by means of a respiration system with or without a forehead support, it is necessary to have two different respiration systems.

### SUMMARY OF THE INVENTION

The object of the present invention is therefore to provide a respiration system that can be adapted to the greatest variety of anatomies and requirements of a patient's head. The respiration system shall be simple and reliable to use and have low manufacturing costs.

This object is accomplished with a respiration system, comprising a breathing mask, a connection means embodied on the breathing mask for connecting the breathing mask to a flexible air tube, and preferably a forehead support, a band system for attaching the breathing mask to a head of a patient, an attaching means arranged on the breathing mask and on the forehead support for attaching the band system to the breathing mask and on the forehead support, whereby the forehead support can be detachably connected to the breathing mask, such that the respiration system can be used for respirating patients with and without a forehead support. The forehead support can be detachably connected to the breathing mask, i.e., the forehead support can be replaced. Different forehead supports, especially forehead supports with a different size, can also be attached to the breathing mask. The respiration system may be used without a forehead support as well, for example, when the patient has head injuries in the area of the forehead. The size of the breathing mask is designed to the effect that the nose and mouth or only the mouth or only the nose can be covered by same and artificially respirated therewith.

In another embodiment, the breathing mask has an attaching means that comprises at least three, and preferably four, attaching elements for the band system. The at least three attaching elements for the band system, i.e., for bands of the band system, are necessary because the respiration system may also be used without a forehead support, i.e., only with the breathing mask on the face, and for this a three-point mounting of the breathing mask is necessary for a secure hold of the breathing mask on the face of the patient.

In a variant, the band system comprises a forehead band.

The forehead band is especially arranged essentially in a plane of symmetry of the respiration system, especially of the breathing mask.

In an additional embodiment, an attaching means for the forehead band is embodied on the forehead support for attaching the forehead band to the forehead support when the respiration system is used without forehead support.

In a complementary embodiment, a connection means, e.g., a hook and loop fastener or a button, is embodied on the forehead band for attaching a front end of the forehead band to a rear area of the band system, and especially in the area of a surface element, when the respiration system is used without the forehead support.

In another embodiment, an attaching means for the forehead band is embodied on the breathing mask for attaching the forehead band to the breathing mask when the respiration system is used without the forehead support.

The forehead band can thus be attached both to the forehead support and to the breathing mask depending on whether the respiration system is used with or without a forehead support.

In a complementary variant, the forehead support is attached to the breathing mask by means of a positive-locking and/or nonpositive-locking fixing means. The forehead support or a component of the forehead support may also be attached to the breathing mask to the extent that the forehead support or component of the forehead support is connected to the breathing mask in a nonadjustable way, e.g., rigidly, with the fixing means in the relative position to the breathing mask. A position adjustment of components of the forehead support, especially of components that lie on the head of the patient, is possible with other devices, e.g., a slot with catches or a fixable hinge for adaptation to different anatomies of the head of the patient.

The fixing means is advantageously a snap-in or snap connection.

In another embodiment, the fixing means may also be used as an attaching means for attaching the band system, especially the forehead band. The fixing means may thus be used both for attaching the band system and for fixing the forehead support.

In a complementary variant, the attaching means and/or fixing means are embodied as a loop or a slot.

In a variant, a second end of the forehead support is provided with a hook for hanging in the fixing means. The hook may be hung, for example, in the slot on the breathing mask. The forehead support is consequently connected pivotably on the breathing mask for adaptation to different anatomies of the patient.

The forehead support and/or breathing mask consist preferably at least partly of plastic.

In another embodiment, the band system comprises bands, which are connected to one another at an end with a net or a surface element or a connecting piece. The net, surface element or connecting piece lie of the back of the head of the patient.

The band system is advantageously elastic for adaptation to the head of the patient and for applying a force for pressing the breathing mask and optionally the forehead support on the patient's head.

In another variant, the position of the forehead support can be adjusted, especially fixed, relative to the breathing mask. The forehead support or a part or component of the forehead support can thus be adjusted in position relative to the breathing mask. This can be accomplished, for example, by means of a slot with catches, by a part or component of the forehead support being movable in a lockable manner. Moreover, this can be done with a fixable hinge, by means of which a component of the forehead support or the forehead support can be set or fixed in various positions relative to the breathing mask. Insofar as the entire forehead support can be moved with the hinge in relation to the breathing mask, the hinge may also be used as a fixing means for fixing the forehead support to the breathing mask.

The band system preferably consists at least partly of a fabric, especially of plastic fibers.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a lateral view of a respiration system with a forehead support;

FIG. 2 is a front view of the respiration system according to FIG. 1;

FIG. 3 is a top view of a band system; and

FIG. 4 is a lateral view of the respiration system without the forehead support.

### DESCRIPTION OF THE PREFERRED EMBODIMENTS

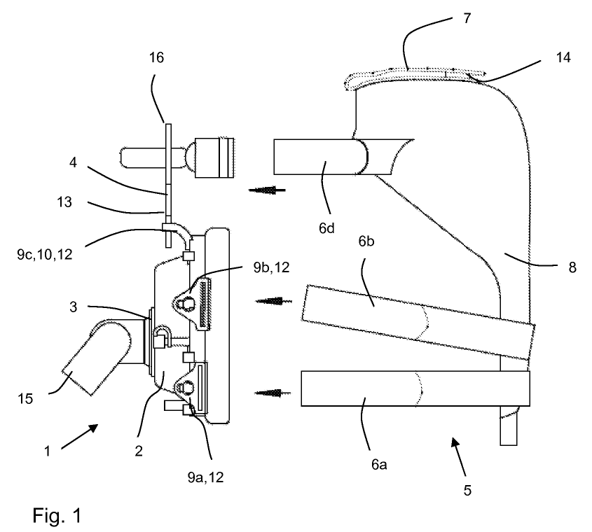

Referring to the drawings in particular, a respiration system 1 is used for the noninvasive artificial respiration of patients. The respiration system 1 comprises a breathing mask 2 with a connection means 3 for a flexible air tube 15 for the detachable fixing of the flexible air tube 15 to the breathing mask 2, a forehead support 4 with a padding (not shown) for application to the forehead, a band system 5 and attaching means 9a, 9b, 9c for the band system 5 arranged on the breathing mask (FIG. 1).

Because of its geometry, the breathing mask 2 can cover both the nose and mouth of a patient, i.e., can artificially respirate the nose and the mouth at the same time. The breathing mask 2 is attached to the patient's head (not shown) by means of the elastic band system 5.

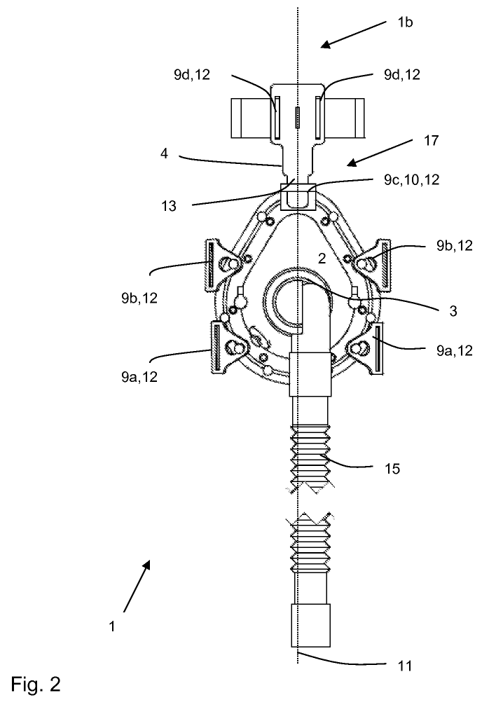

On the breathing mask 2, there are two lower attaching means 9a and two upper attaching means 9b, which are each embodied as loops 12 (FIG. 2). Another loop 12 is present as a fixing means 10 at the upper end of the breathing mask 2. At a first end 16, the forehead support 4 has two lateral attaching means 9d as loops 12. At a second end 17, the forehead support 4 is provided with a hook 13 (FIGS. 1, 2 and 4).

The band system 5 has two lower bands 6a, two middle bands 6b, two upper bands 6d and a forehead band 7. At each of the ends of the bands 6a, 6b, 6d and of the forehead band 7 is embodied a loop (not shown), by means of which the bands 6a, 6b, 6d can be attached in the loops 12.

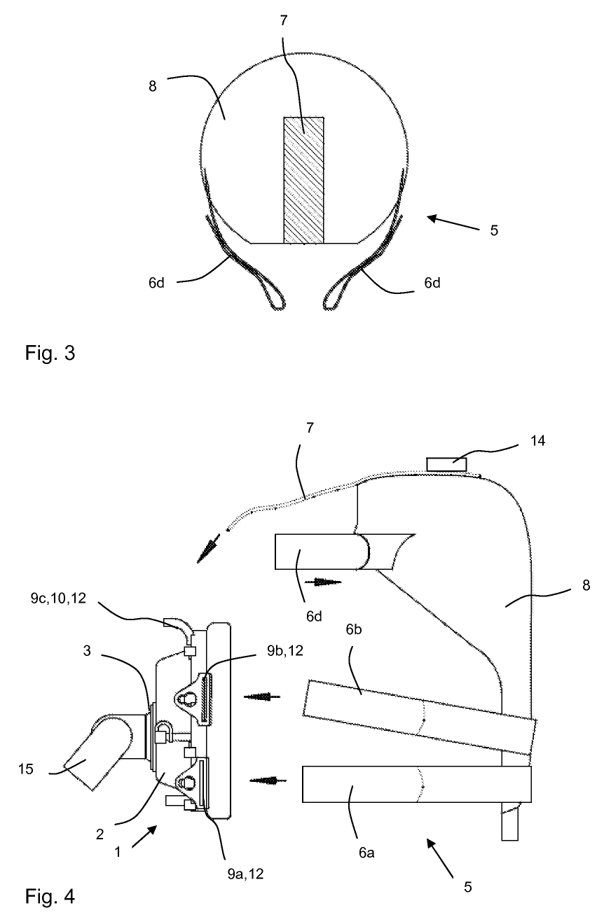

When the respiration system 1 is used without the forehead support 4, the end of the forehead band 7 is detachably attached with the loop by means of a hook and loop fastener 14 at another end of the forehead band 7 in the area of a surface element 8 and thus not used. In a (not shown) embodiment, the end of the forehead band 7 may also be attached to the upper end 16 of the forehead support 4, such that a hook and loop fastener 14 is not necessary. The other end of the bands 6a, 6b, 6d and of the forehead band 7 is not detachably attached to the surface element 8 embodied as a net (FIG. 1). In this case, the two lower bands 6a are attached to the forehead support 4 in the two lower attaching means 9a, the two middle bands 6b in the two upper attaching means 9b and the two upper bands 6d in the two lateral attaching means 9d. The forehead band 7 is folded back by means of a hook and loop fastener 14 and attached to the forehead band 7 or to the surface element 8 (FIGS. 1 and 3). The hook 13 of the forehead support is hung in the fixing means 10 embodied as a loop 12. Thus, the respiration system 1 with a forehead support 4 can be placed on the head of a patient. The forehead support 4 is pivotable about the fixing means 10 embodied as a loop 12, because the hook 13 is hung into the loop 12, such that the respiration system 1 can be adapted to different anatomies of heads.

Moreover, the respiration system 1 may also be used without the forehead support 4, e.g., in case of an injury of the patient's forehead (FIG. 4). To this end, only the forehead support 4 needs to be removed from the fixing means 10 embodied as a loop 12 and the front end of the forehead band 7 needs to be detached from the hook and loop fastener 14 and hung on the fixing means 10. The loop 12 embodied at the upper end of the breathing mask 2 is thus used both as attaching means 9c for attaching the forehead band 7 and for fixing the forehead support 4 by means of the hook 13. When used without the forehead support 4, the upper bands 6d are attached either to the upper attaching means 9b or attached to the surface element 8 by means of a hook and loop fastener 14 in the area of the end of the upper bands 6d, i.e., not used (not shown). Similar to the use with the forehead support 4, the bands 6a, 6b are attached to the attaching means 9a, 9b.

All in all, considerable advantages are connected with the respiration system 1 according to the present invention. The respiration system 1 can be used in a simple manner both with and without a forehead support 4 for respirating patients, so that only one respiration system 1 must be available for adaptation to patients.

While specific embodiments of the invention have been shown and described in detail to illustrate the application of the principles of the invention, it will be understood that the invention may be embodied otherwise without departing from such principles.

### LIST OF REFERENCE NUMBERS

1 Respiration system
2 Breathing mask
3 Connection means
4 Forehead support
5 Band system
6 Band (6a, 6b, 6d)
7 Forehead band
8 Surface element
9 Attaching means (9a, 9b, 9c, 9d)
10 Fixing means
11 Plane of symmetry
12 Loop
13 Hook
14 Hook and loop fastener
15 Flexible air tube
16 First end of the forehead support
17 Second end of the forehead support

What is claimed is:

1. A respiration system comprising:
a breathing mask;
a connection means embodied on the breathing mask for connecting the breathing mask to a flexible air tube;
a forehead support;
a band system for attaching the breathing mask to a head of a patient; and
an attaching means for attaching the band system to the breathing mask, the attaching means being arranged on at last one of the breathing mask and on the forehead support, said forehead support being detachably connected to the breathing mask whereby the respiration system can be used for respirating patients both with and without said forehead support.

2. A respiration system in accordance with claim 1, wherein the attaching means comprises at least three attaching elements for attaching the band system to the breathing mask.

3. A respiration system in accordance with claim 1, wherein the band system comprises a forehead band.

4. A respiration system in accordance with claim 3, wherein the forehead band is essentially arranged in a plane of symmetry of the breathing mask.

5. A respiration system in accordance with claim 3, further comprising a connection means on the forehead band for attaching a front end of the forehead band to a rear area of the band system in the area of a surface element, when the respiration system is used without the forehead support.

6. A respiration system in accordance with claim 3, wherein on the breathing mask the attaching means comprises an attaching element for the forehead band embodied for attaching the forehead band to the breathing mask when the respiration system is used without the forehead support.

7. A respiration system in accordance with claim 1, further comprising a detachable connection including a positive-locking and/or nonpositive-locking fixing means detachably fixing the forehead support to the breathing mask.

8. A respiration system in accordance with claim 7, wherein the fixing means is a snap-in or snap connection.

9. A respiration system in accordance with claim 7, wherein the fixing means further forms a part of said attaching means for attaching a forehead band of the band system.

10. A respiration system in accordance with claim 7, wherein the attaching means and/or the fixing means is embodied as a loop or a slot.

11. A respiration system in accordance with claim 10, wherein a second end of the forehead support is provided with a hook for hanging in the fixing means.

12. A respiration system in accordance with claim 1, wherein the forehead support and/or the breathing mask is formed at least partly of plastic.

13. A respiration system in accordance with claim 1, wherein the band system comprises bands connected to one another with an end with a net or a surface element or a connecting piece.

14. A respiration system in accordance with claim 1, wherein the band system is elastic.

15. A respiration system in accordance with claim 1, wherein the position of the forehead support can be adjusted, and can be especially fixed, relative to the breathing mask.

16. A respiration system comprising:
a breathing mask;
a connection means embodied on the breathing mask for connecting the breathing mask to a flexible air tube;
a forehead support;
a band system for attaching the breathing mask to a head of a patient;
attaching elements attaching the band system to the breathing mask, the attaching elements being arranged on at last one of the breathing mask and on the forehead support; and
a detachable connection between said breathing mask and said forehead support, for detachably connecting the forehead support to the breathing mask whereby the respiration system can be used for respirating patients both with and without a forehead support.

17. A respiration system in accordance with claim 1, wherein the band system comprises a forehead band.

18. A respiration system in accordance with claim 17, further comprising a connection means on the forehead band for attaching a front end of the forehead band to a rear area of the band system in an area of a surface element, when the respiration system is used without the forehead support.

19. A respiration system in accordance with claim 17, wherein:
the detachable connection comprises a positive-locking and/or nonpositive-locking fixing means detachably fixing the forehead support to the breathing mask; and
the attaching elements include an attaching element for the forehead band embodied for attaching the forehead band to the breathing mask when the respiration system is used without the forehead support.

20. A respiration system in accordance with claim 19, wherein the fixing means is a snap-in or snap connection and the fixing means further forms a part of said attaching means for attaching said forehead band of the band system.
